(19) United States
(12) Patent Application Publication
(10) Pub. No.: US 2024/0024616 A1
(43) Pub. Date: Jan. 25, 2024

(54) SLEEP DISTURBANCE CHAMBER FOR ANIMAL TEST SUBJECTS

(71) Applicant: UNIVERSITY OF SOUTH CAROLINA, COLUMBIA, SC (US)

(72) Inventors: MATTHEW EFIRD, COLUMBIA, SC (US); CONRAD M. GORE, COLUMBIA, SC (US); ANA POCIVAVSEK, COLUMBIA, SC (US); HOMAYOUN VALAFAR, ELGIN, SC (US)

(21) Appl. No.: 18/353,326

(22) Filed: Jul. 17, 2023

Related U.S. Application Data
(60) Provisional application No. 63/390,883, filed on Jul. 20, 2022.

(51) Int. Cl. A61M 21/00

(52) U.S. Cl. CPC A61M 21/0094; A61M 2021/0083; A61M 2250/00

(57) ABSTRACT

The disclosure deals with a system and method for inducing sleep loss in animal test subjects, such as rats. A sleep deprivation chamber uses a DC Servo motor with a controller to cause a bar to sweep across the rodent cage floor. The system is programmed to sweep the bar across the floor of the rodent cage at varying intervals. The maximum speed at which the bar sweeps the entire rodent cage is within 3 seconds. The chamber is designed such that it can structurally accommodate a receiver and record EEG/EMG telemetrically in animals that have been implanted with sleep transmitters. During the sleep deprivation protocol, food and water are available to animals in the chamber at all times. The chamber is also designed to fit a standardized rodent (e.g., rat or mouse) cage, and plural cages such as an 8-chamber sleep restriction device can be simultaneously operated.

### PRIORITY CLAIM

The present application claims the benefit of priority of U.S. Provisional Patent Application No. 63/390,883, titled Sleep Disturbance Chamber For Animal Test Subjects, filed Jul. 20, 2022, and which is fully incorporated herein by reference for all purposes.

### STATEMENT REGARDING FEDERALLY SPONSORED RESEARCH OR DEVELOPMENT

This invention was made with government support under Grant No. RO1 NS102209, awarded by National Institute of Neurological Disorders and Stroke (NINDS), an Institute of the National Institutes of Health, and Grant No. 2P20GM103499-20, awarded by the National Institute of General Medical Sciences (NIGMS), an Institute of the National Institutes of Health (NIH). The government has certain rights in the invention.

### BACKGROUND OF THE PRESENTLY DISCLOSED SUBJECT MATTER

This disclosure deals with a system and method for inducing sleep loss in animal test subjects such as rodents (e.g., rats and mice). In particular, disclosed subject matter allows investigators to sleep deprive multiple subjects in parallel with an at least partially automated system.

In various animal research protocols, it is necessary to deprive rats or mice of sleep. Some estimates indicate as many as about 3,000 basic science sleep researchers in the United States alone. A common method which has been used by researchers simply involves the researcher gently handling a test subject to keep it awake. However, it is difficult with such approach to simultaneously sleep deprive plural subjects in a way which provides consistent stimulation over time simultaneously in parallel to plural subjects. Further, it is desirable to not stimulate an animal with novel objects that could induce behavioral plasticity.

As experimenters, even if using a gentle handling approach, it is often difficult to accurately sleep deprive more than 2 animals at one time given that towards the latter end of the protocol (e.g., 5 to 6 hours), animals are quite tired and are difficult to keep awake. An automated system would alleviate such discrepancies and also allow users to sleep deprive multiple animals at the same time, if using multiple chambers.

Summary of the Presently Disclosed Subject Matter

The presently disclosed subject matter deals with a system and method for inducing sleep loss in animal test subjects such as rats or mice. A sleep deprivation chamber uses a DC Servo motor with a controller to cause a bar to sweep across the rodent cage floor. The system is programmed to sweep the bar across the floor of the rodent cage at varying intervals. The maximum speed at which the bar sweeps the entire rodent cage may be a set time, for example, such as within 3 seconds. The chamber is designed such that it can structurally accommodate a receiver and can record EEG/EMG telemetrically in animals that have been implanted with sleep transmitters. During the sleep deprivation protocol, food and water are available to animals in the chamber at all times. The chamber is also designed to fit a standardized rat cage, and plural cages, such as an 8-chamber sleep restriction device, can be simultaneously operated.

The presently disclosed animal protocol allows us to use plural chambers to deprive a plurality of rats of sleep. Some advantages of such chambers over, for example, a currently used gentle handling method:
does not stimulate the animal with novel objects that induce behavioral plasticity;
animals are not handled by experimenter during sleep deprivation;
automated deprivation system; and
allows for simultaneous sleep deprivation of multiple animals in parallel chambers.

In one exemplary embodiment disclosed herewith, methodology for inducing sleep loss in animal test subjects may preferably comprise providing an animal enclosure cage with a removable lid forming an elongated slot along the top of the enclosure cage; mounting a controllably movable arm adjacent to the cage; positioning the movable arm for projection into the cage through the elongated slot so that at least a portion of the arm is movable adjacent to the floor of the cage; and controllably moving the movable arm along the cage floor so that an animal test subject in the cage is periodically moved to deprive sleep of the animal test subject.

It is to be understood that the presently disclosed subject matter equally relates to associated and/or corresponding systems.

Other example aspects of the present disclosure are directed to systems, apparatus, tangible, non-transitory computer-readable media, user interfaces, memory devices, and electronic devices related to sleep deprivation. To implement methodology and technology herewith, one or more processors may be provided, programmed to perform the steps and functions as called for by the presently disclosed subject matter, as will be understood by those of ordinary skill in the art.

Another exemplary embodiment of presently disclosed subject matter relates to a system for inducing sleep loss in animal test subjects, preferably comprising a removable lid configured to fit on a preexisting animal enclosure cage, with an elongated slot formed along the top of the enclosure cage; a guide rail situated adjacent to the cage; a movable arm received on the guide rail for movement there along; and a controllable motor for controllably driving the arm relative to the guide rail. Per such exemplary system, preferably the movable arm is an L-shaped arm having at least a portion thereof which is positioned to extend through the elongated slot and along the width of the cage floor while the arm is received on the guide rail, so that controllably moving the arm through operation of the controllable motor moves the movable arm along the cage floor so that an animal test subject in the cage is periodically moved to deprive sleep of the animal test subject.

Yet another exemplary embodiment of presently disclosed subject matter relates to an automated system for inducing sleep loss in rat test subjects according to a programmed sleep deprivation protocol. Preferably, such exemplary automated system may comprise a rat enclosure cage; a removable lid configured to fit on the cage, with an elongated slot formed along the top of the cage; a guide rail supported adjacent to the cage; a movable sweep arm received on the guide rail for movement back and forth along the rail; a controllable motor for controllably driving the sweep arm relative to the guide rail; and a programmable motor controller for controlling operation of the motor according to a programmed sleep deprivation protocol. Further, preferably, such sweep arm is L-shaped with a base portion thereof positioned to extend through the elongated slot and along the width of the cage floor while the arm is received on the guide rail, so that controllably moving the arm through operation of the controllable motor moves the movable arm along the cage floor so that a rat test subject in the cage is periodically moved to deprive sleep of the rat test subject according to the programmed sleep deprivation protocol.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summary section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, will appreciate that the presently disclosed subject matter applies equally to corresponding methodologies as associated with practice of any of the present exemplary devices and vice versa.

These and other features, aspects and advantages of various embodiments will become better understood with reference to the following description and appended claims. The accompanying figures, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a perspective view of an exemplary single chamber implementation of presently disclosed subject matter, including a controlled movable arm, and associated monitor for the movable arm control system;

FIG. 2 illustrates in chart format one presently disclosed exemplary control protocol for sleep deprivation of subjects;

FIGS. 3A-3D, respectively, graphically illustrate Sleep Deprivation Chamber Results regarding use of the FIG. 1 exemplary embodiment, in particular with FIG. 3A illustrating Wake Duration data, FIG. 3B illustrating rapid eye movement (REM) Duration data, FIG. 3C illustrating non-rapid eye movement (NREM) Duration Data, and FIG. 3D illustrating data representing impact of sleep deprivation;

FIG. 4 illustrates a generally front perspective view of an exemplary embodiment of an 8-chamber arrangement in accordance with presently disclosed subject matter;

FIG. 5A illustrates a front view of a monitor screen, representing control features of corresponding presently disclosed subject matter;

FIG. 5B illustrates a front view of the monitor screen when showing an accessed Delay Run Timer of presently disclosed subject matter;

FIG. 6A illustrates a perspective view of an exemplary Plastic Cage Cover in accordance with presently disclosed subject matter;

FIG. 6B illustrates a relatively enlarged, generally top view of the exemplary cover of subject FIG. 6A;

FIG. 7A illustrates a perspective view of an exemplary Motor and related/associated features of presently disclosed subject matter;

FIG. 7B illustrates a relatively enlarged image of a Home Switch feature in accordance with presently disclosed subject matter;

FIG. 7C illustrates a perspective view of an exemplary Guide Rail in accordance with presently disclosed subject matter;

FIG. 7D illustrates a perspective view of an exemplary presently disclosed Carriage and Sweeping Arm arrangement in accordance with presently disclosed subject matter;

FIG. 8 illustrates a perspective view of exemplary presently disclosed motor controller features;

FIG. 9 illustrates a perspective view of an exemplary programmable logic controller (PLC) Board, for use in accordance with presently disclosed subject matter;

FIG. 10A illustrates a generally perspective view of exemplary Power Box features, particularly representing power banks thereof, for use in accordance with presently disclosed subject matter;

FIG. 10B illustrates a generally perspective view of exemplary Power Box features, particularly representing power and ground bus bars thereof, for use in accordance with presently disclosed subject matter;

FIGS. 11A-11B respectively represent perspective views of differently colored colorized flashing indicators relating to motor operations, in accordance with presently disclosed subject matter;

FIGS. 12A-12C respectively illustrate perspective views of progress positions in the process of a user removing a Metal Door from a Plastic Cage Cover, according to presently disclosed subject matter, particularly illustrating positioned (FIG. 12A), partially removed (FIG. 12B), and fully removed (FIG. 12C) positions thereof;

FIGS. 12D-12E respectively illustrate perspective views showing raising of a Support Arm feature to allow the associated cage to slid into place;

FIG. 12F illustrates a perspective view representing an associated cage sliding into the system until it hits the Cage Locating Pin features thereof;

FIG. 12G illustrates a perspective view representing the Support Arm lowered to its lowered position;

FIG. 12H illustrates a perspective view representing the Metal Door feature put back on the associated cage and the system readies for experiments;

FIG. 13A illustrates a front view of a monitor screen, representing Motor Selection Option control features of corresponding presently disclosed subject matter;

FIG. 13B illustrates an isolated enlarged front view of a portion of the monitor screen of FIG. 13A, as an option for programing by a user to determine how many motors are allowed to run during an experiment;

FIG. 13C illustrates an isolated enlarged front view of a portion of the monitor screen of FIG. 13A, as a further portion of an option for programing by a user to determine how many motors are allowed to run during an experiment;

FIG. 14A illustrates a front view of a monitor screen, representing Delay Option control features of corresponding presently disclosed subject matter Option

FIG. 14B illustrates a front view of the monitor screen of FIG. 14A as a further portion of a delay option for use by a user, for the user to enter in the time of the desired delay period;

FIG. 14C illustrates a front view of the monitor screen of FIG. 14A as a further portion of a delay option for use by a user, for the user to check whether the program has started;

FIG. 14D illustrates a front view of the monitor screen of FIG. 14A as a further portion of a delay option for use by a user, for the user to check a timer which states how long the delay has been running;

FIG. 15A illustrates a front view of a monitor screen of presently disclosed subject matter as a further portion of an option for use by a user, for the user to enter a Program, in particular so that the user may enter in the Total Run Time;

FIG. 15B illustrates a front view of a monitor screen of FIG. 15A as a further portion of an option for use by a user, for the user to select how many segments are needed by pressing the Duration Selection Buttons;

FIG. 15C illustrates a front view of a monitor screen of presently disclosed subject matter as a further portion of an option for use by a user, for the user to select options associated with operations once the Duration Selection Button is pressed;

FIG. 15D illustrates an isolated enlarged front view of a portion of the monitor screen of FIG. 15C, as a further portion of an option for programing by a user to select what Time Value is needed for the Duration Time by pressing the Duration Time Value Selection Button shown in FIG. 15D;

FIG. 15E illustrates an isolated enlarged front view of a portion of the monitor screen of FIG. 15C, as a further portion of an option for programing by a user to select what Hours Time Value is needed for the Duration Time by pressing the Hours Duration Time Value Selection Button shown in FIG. 15E;

FIG. 15F illustrates an isolated enlarged front view of a portion of the monitor screen of FIG. 15C, as a further portion of an option for programing by a user to select what Minutes Time Value is needed for the Duration Time by pressing the Minutes Duration Time Value Selection Button shown in FIG. 15F;

FIG. 15G illustrates an isolated enlarged front view of a portion of the monitor screen of FIG. 15C, as a further portion of an option for programing by a user to select the amount of time the Duration will last for the Duration Length Entry Location;

FIG. 15H illustrates an isolated enlarged front view of a portion of the monitor screen of FIG. 15C, as a further portion of an option for programing by a user to select what Time Value is needed for the Pause Time, which value can be changed by pressing on the Pause Time Value Selection Button shown in FIG. 15H;

FIG. 15I illustrates an isolated enlarged front view of a portion of the monitor screen of FIG. 15C, as a further portion of an option for programing by a user to select what Minutes Time Value is needed for the Pause Time by pressing the Minutes Pause Time Value Selection Button shown in FIG. 15I;

FIG. 15J illustrates an isolated enlarged front view of a portion of the monitor screen of FIG. 15C, as a further portion of an option for programing by a user to select what Seconds Time Value is needed for the Pause Time by pressing the Seconds Pause Time Value Selection Button shown in FIG. 15J;

FIG. 15K illustrates an isolated enlarged front view of a portion of the monitor screen of FIG. 15C, as a further portion of an option for programing by a user to select the amount of time the Pause will last for the Pause Length Entry Location;

FIG. 16A illustrates a front view of a monitor screen of FIG. 5A as a further portion of an option for use by a user, for the user to confirm entry of Values and that the program is ready to Start, and subsequently check either the Program Running Timer or the Delay Timer if a delay is set to determine the program has started because either Timer is counting up;

FIG. 16B illustrates a front view of a monitor screen of presently disclosed subject matter as a further portion of an option for use by a user, for the user to confirm that the program has started because either Timer is counting up; and

FIG. 16C illustrates a perspective view of a presently disclosed exemplary embodiment in or after use and of indicating points (either indicated Point A or indicated Point B) as points at which motor movement will eventually terminate if the program is stopped or has finished.

Repeat use of reference characters in the present specification and figures is intended to represent the same or analogous features or elements of the present invention.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

In general, the present disclosure is directed to a system and method for inducing sleep loss in animal test subjects, such as rats. A sleep deprivation chamber uses a controlled motor to cause a bar to sweep across the rodent cage floor at varying intervals. The chamber or enclosure is designed such that it can structurally accommodate a receiver and can record EEG/EMG telemetrically in animals that have been implanted with sleep transmitters. Food and water remain fully available to animals at all times in the chamber. In some embodiments, a presently disclosed chamber may be also designed to fit a standardized rat cage. In other embodiments, plural cages such as an 8-chamber sleep restriction device can be simultaneously operated.

To induce sleep loss in rats, we have designed a sleep deprivation chamber using for example a Teknic™ 24v DC Servo motor (Teknic, Inc., Victor, NY) that operates with for example a Velocio PLC (Velocio Networks, Huntsville, Alabama) to sweep a bar across the rodent cage floor. The programmable logic controller (PLC) may be programmed for example with Velocio vBuilder software, an open source coding program, and use, for example, a Velocio monitor that is connected via HDMI. The system may be programmed to sweep the bar across the floor of the rodent cage at varying intervals. The maximum speed at which the bar sweeps the entire rodent cage may be set, for example, such as within 3 seconds. The chamber is designed such that we can place it over a Data Science International (DSI; a division of Harvard Bioscience, Inc.; Holliston, MA) receiver and record EEG/EMG telemetrically in animals that have been implanted with sleep transmitters. During the sleep deprivation protocol (e.g., 6 hours), food and water are available to animals in the chamber at all times. The chamber is also designed to fit a standard rat cage from veterinary resources and cages can be replaced upon completion of an experiment.

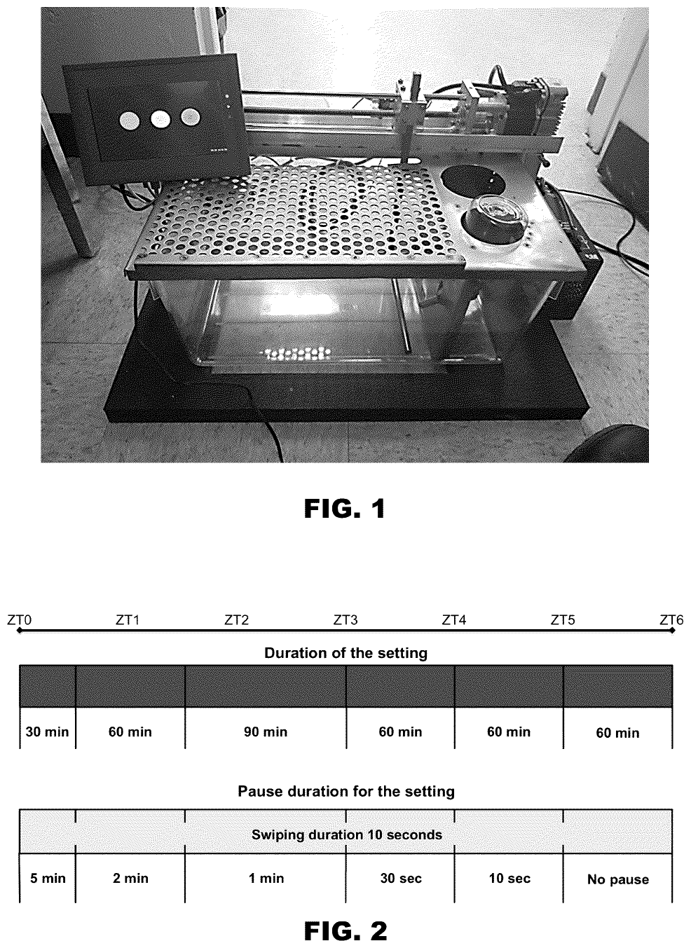

FIG. 1 illustrates an exemplary single chamber implementation of presently disclosed subject matter, including a controlled movable arm, and associated monitor for the movable arm control system.

Plural subjects may be addressed in parallel. For example, we have developed one exemplary embodiment which comprises an 8-chamber sleep disturbance apparatus. It is functional for use with rodents as shown by feasibility testing. For example, such 8-chamber sleep restriction chamber can be used by neuroscientists to prevent small animals, such as rats and mice, from initiating into sleep. The chamber works in an automated fashion so that it sweeps a bar across the floor of the rodent cage. This will physically nudge the animal and prevent it from falling asleep. Thus, it allows, in automated fashion, to sleep restrict and sleep deprive a plurality of small animals and test hypotheses related to the role of sleep.

The presently disclosed chambers are designed to work with a basic sleep monitoring system that many neuroscientists use (e.g., the DSI telemetric EEG/EMG system). The chambers can also be programmed to work simultaneously and increase throughput. As compared with stand-alone models, the multi-chamber models disclosed herewith increase efficiency, improve accuracy, and increase productivity. In addition, the presently disclosed technology facilitates incorporation of Artificial Intelligence routines to autonomously operate without the need for constant human intervention.

One presently disclosed exemplary control protocol for sleep deprivation of subjects is represented by FIG. 2.

In particular, another Sleep Deprivation Chamber Protocol may be represented as follows:
1st hour: arm will sweep (speed: 10 sec to sweep entire cage) every 5 minutes
2nd hour: arm will sweep every 3 minutes
3rd hour: arm will sweep every 2 minutes
4th hour: arm will sweep every 1 minute
5th hour: arm will sweep every 30 seconds
6th hour: arm will sweep continuously across the chamber; speed will remain 10 sec to sweep entire cage

For another exemplary protocol arrangement, one can incorporate the use of the sleep deprivation chamber (FIG. 1) into the protocol. This is done preferably for use with rats that have undergone EEG/EMG transmitter surgery to simultaneously collect sleep-wake data and to assess the accuracy of the sleep deprivation protocol. Further, given various previous findings with gentle handling, acute sleep deprivation of 6 hours does not significantly elevate plasma corticosterone in male rats, but it does elevate corticosterone in female rats (Baratta et al. 2018 Scientific Reports). To validate the chamber as an appropriate means to sleep deprive animals, one may take blood collections to assess plasma corticosterone at baseline, 3 h, 6 h, and 24 h after the start of sleep deprivation.

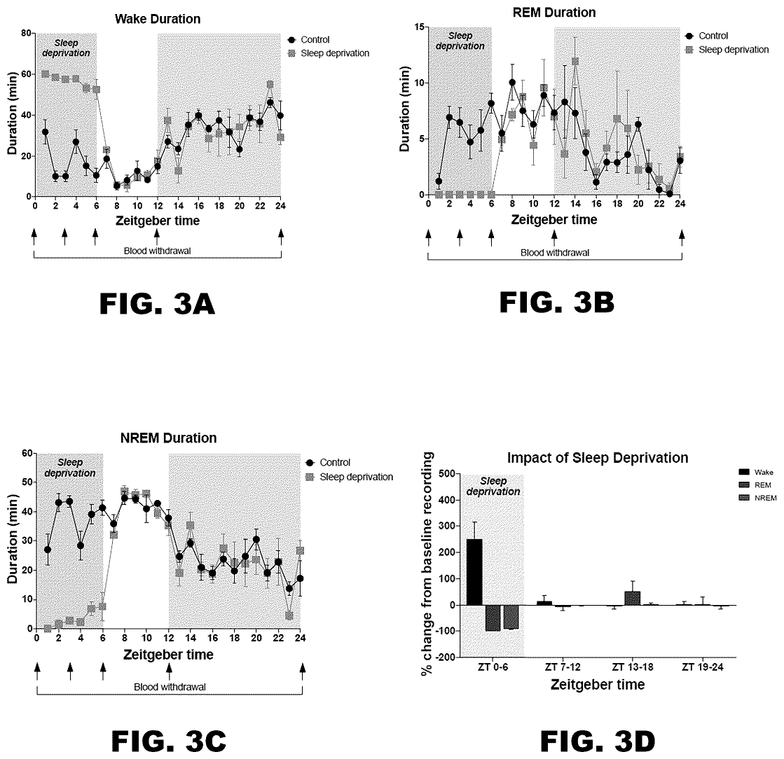

FIGS. 3A through 3D, respectively graphically illustrate Sleep Deprivation Chamber Results regarding use of the FIG. 1 exemplary embodiment. In particular, FIG. 3A illustrates Wake Duration data, FIG. 3B illustrates REM Duration data, FIG. 3C illustrates NREM Duration Data, and FIG. 3D illustrates data representing impact of sleep deprivation.

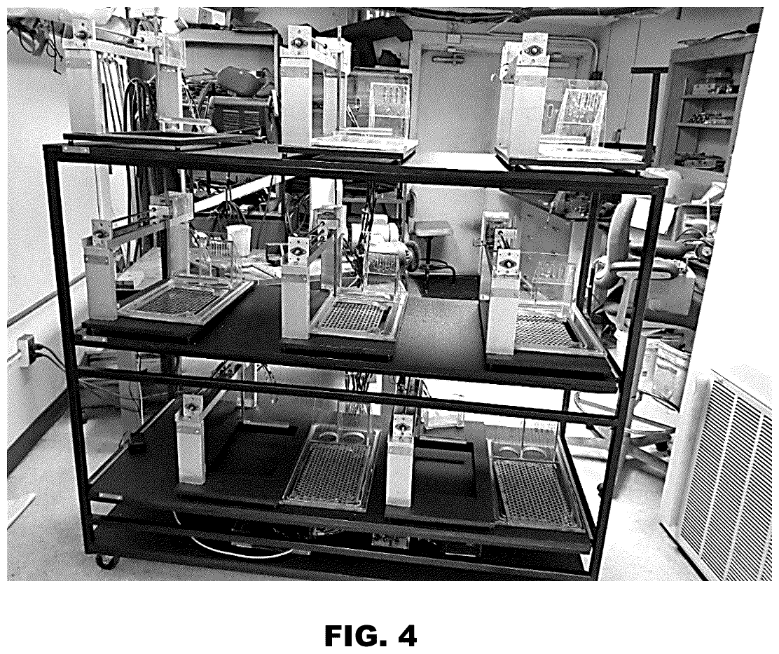

FIG. 4 illustrates an exemplary embodiment of an 8-chamber arrangement in accordance with presently disclosed subject matter, illustrating eight respective rack-mounted separate chambers for simultaneous processing of eight separate subjects in parallel.

### Section 1 Parts List

The following disclosure indicates in effect a “parts” list for such an exemplary embodiment as the associated Monitor and other features of the presently disclosed subject matter.

### Section 1-1 Monitor

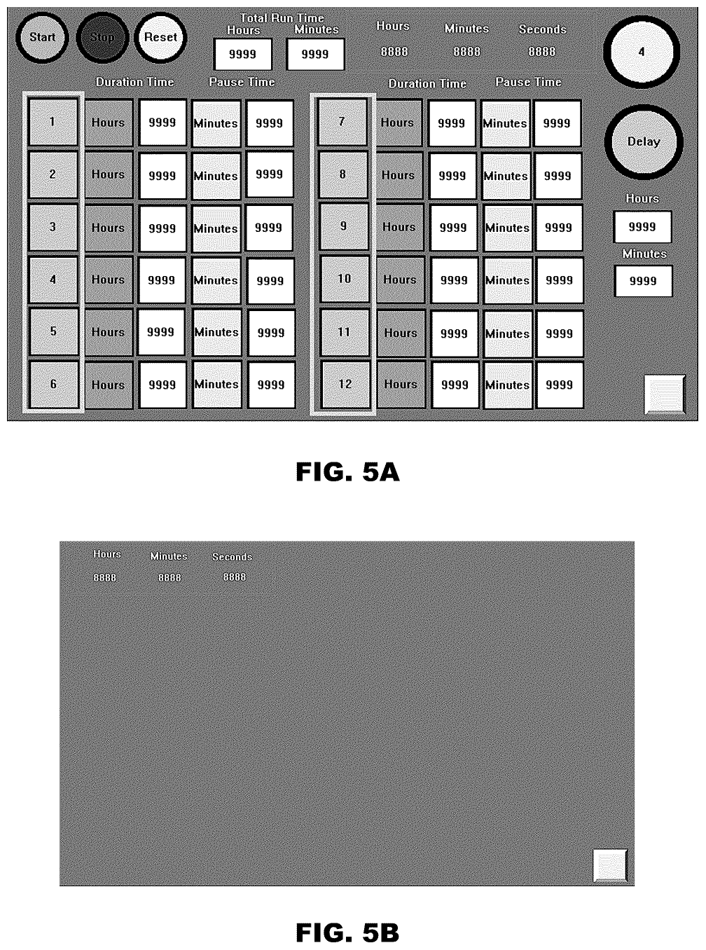

FIG. 5A illustrates a front view of a monitor screen, representing control features of corresponding presently disclosed subject matter. In particular, various features shown include a Start Button, Stop Button, Reset Button, and a Total Run Time Entry Location.

Further, the outline (near right top) in FIG. 5A represents a Program Running Timer. The numbered circular button to the further righthand corner of FIG. 5A represents the Motor Selection functionality. The left and middle outlines in FIG. 5A represent the Duration Selection. Further shown (in the bottom right corner of FIG. 5A), as will be understood by those of ordinary skill in the art, are Duration Time Value Selection, Duration Length Entry Location, Pause Time Value Selection, Pause Length Entry Location, Delay Option Button, Delay Time Entry Location, and Screen Toggle Button.

FIG. 5B (see sole outline) illustrates a front view of the monitor screen when showing the accessed Delay Run Timer.

### Section 1-2 Cage

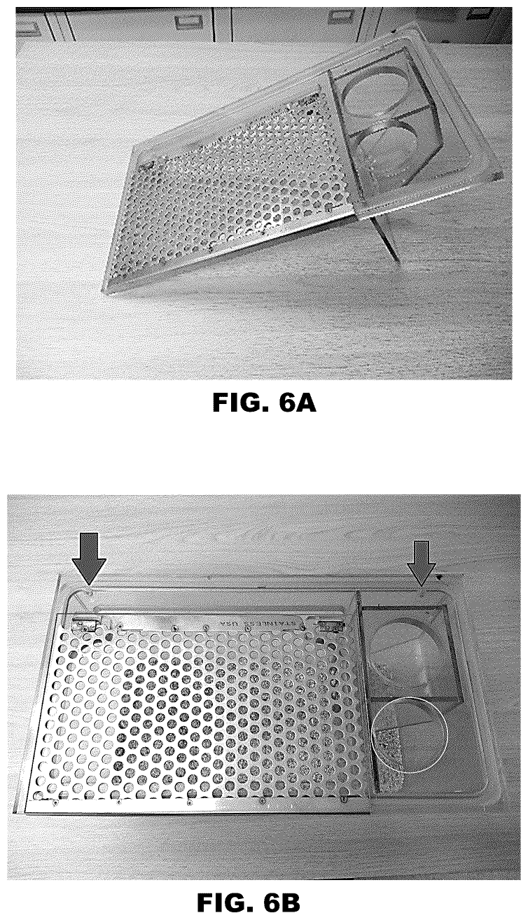

Representing further exemplary features, FIG. 6A illustrates a perspective view of an exemplary Plastic Cage Cover. FIG. 6B illustrates a relatively enlarged, generally top view of such cover, which may be made of plastic and/or other materials. In particular, the upper circle in FIG. 6B illustrates an exemplary Feeder Tray while the lower circle in FIG. 6B illustrates a Water Bottle Holder. The sole rectangle in FIG. 6B illustrates a Metal Door while the two arrows in FIG. 6B represent Location Pin Holes.

### Section 1-3 Motorized Sweeping Arm

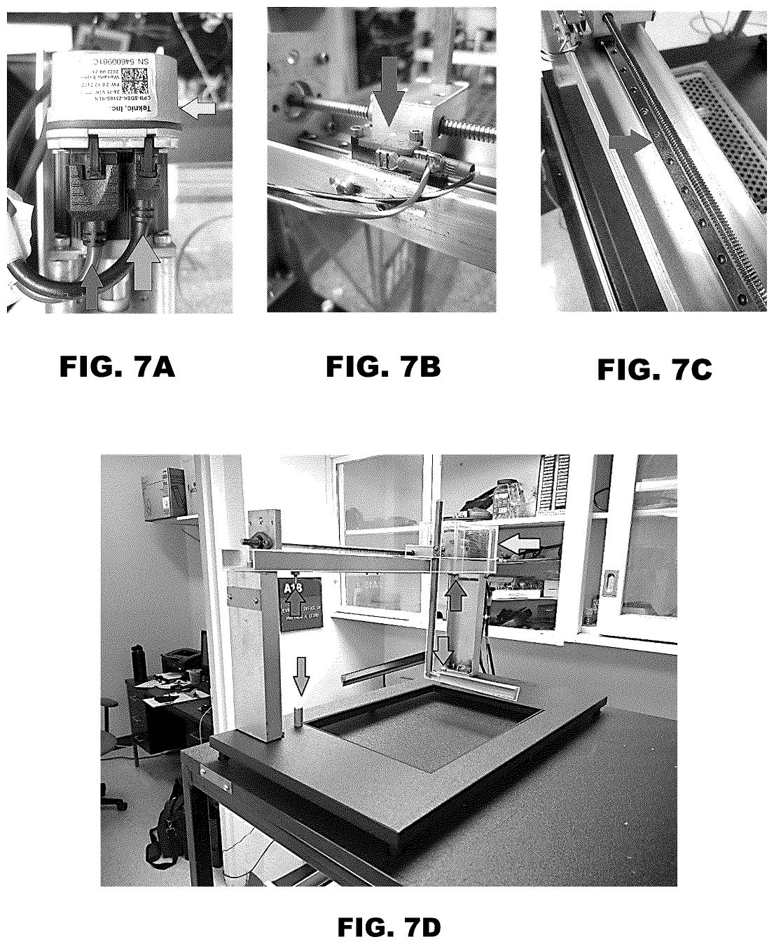

FIG. 7A illustrates a perspective view of an exemplary Motor (see top right arrow) and related/associated features of presently disclosed subject matter. FIGS. 7B and 7C in particular illustrate enlarged images of exemplary associated features. For example, the sole arrow in FIG. 7B points to a Faraday Cage. A Home Switch feature is also otherwise shown in FIG. 7B. The bottom left arrow in FIG. 7A illustrates a Communication Cable, while the bottom right arrow thereof illustrates a Power Cable. Moreover, in particular, FIG. 7C illustrates a perspective view of a Guide Rail.

FIG. 7D is a further perspective view of an exemplary presently disclosed Carriage and Sweeping Arm arrangement, as specified by the right middle outline in FIG. 7D. The upper pointing arrows thereof show Location Pins, while the downward pointing arrows in FIG. 7D point to Cage Locating Pins. The lower outline in FIG. 7D illustrates the Receiver Plate Hole while the presently disclosed Support Arm is within the top flat outline in FIG. 7D.

### Section 1-4 Motor Controllers

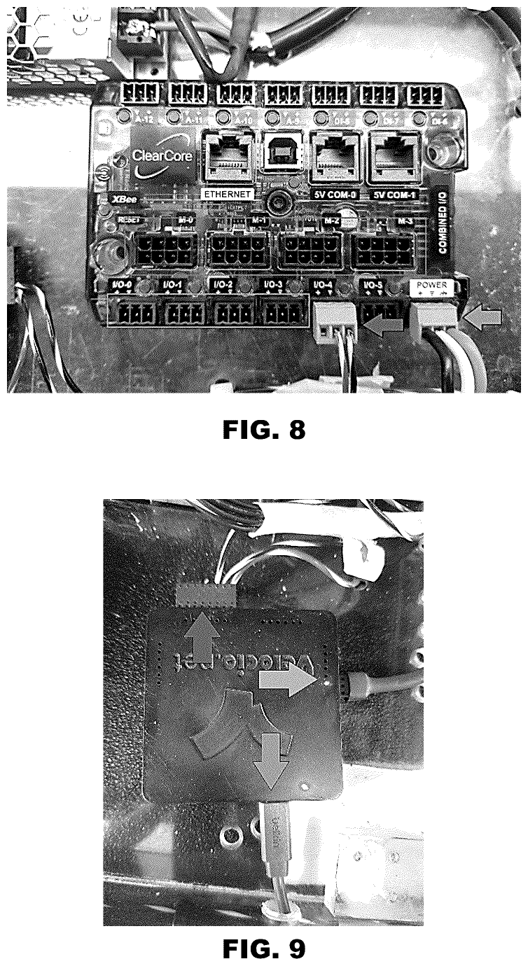

FIG. 8 illustrates a perspective view of exemplary presently disclosed motor controller features, with the right arrow thereof designating the Power Cable. The upper box in FIG. 8 designates the Motor Communication Cable Ports, while the lower box thereof illustrates Home Switch Slots. The left arrow in FIG. 8 points to the Velocio Input Slot. Other brand PLCs and the like may be practiced.

### Section 9 Velocio Board (PLC Board)

FIG. 9 illustrates a perspective view of an exemplary PLC Board, with the lower arrow thereof referring to the Power Cable. The middle arrow in FIG. 9 refers to the Monitor Communication Cable, while the upper arrow thereof refers to the Motor Controller Command Cables.

### Section 1-6 Power Box

FIGS. 10A and 10B illustrate respective generally perspective views of exemplary Power Box features. For example, the left and right arrows, respectively, of FIG. 10A point to Power Bank 1 and Power Bank 2. In FIG. 10B, the far left box designates the Power Bus Bars for Bank 1, while the Power Bus Bars for Bank 2 are designated by the far right box in FIG. 10B. The middle box in FIG. 10B designates the Faraday Cage Ground Bus Bar.

### Section 2 Standard Operation

The following discloses exemplary operation of an exemplary embodiment of the presently disclosed subject matter.

### Section 2-1 Power Up Procedures

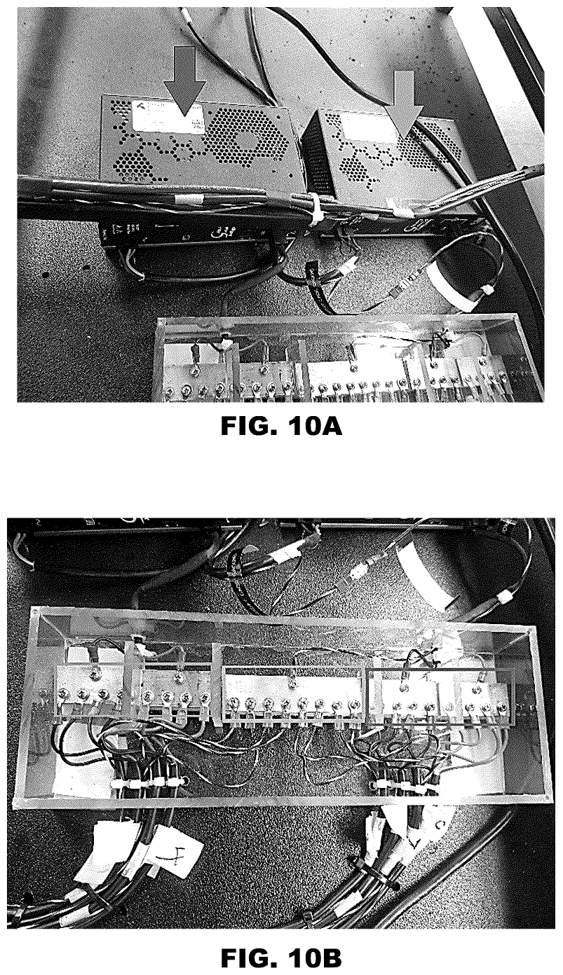

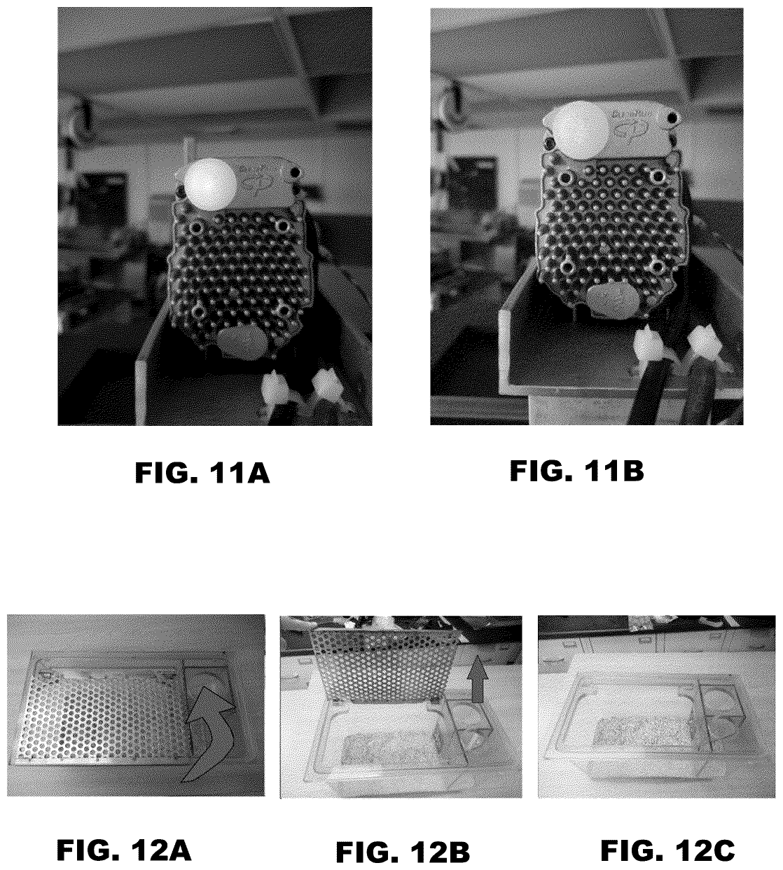

When power is first turned on to the system, the motors will start flashing orange in the back, as represented by FIG. 11A. After a few seconds, motors 1 and 5 will start flashing green, followed by 2 and 6, then 3 and 7, and finally 4 and 8, as represented by FIG. 11B. When all the motors have turned green, motors 1 and 5 will ‘home out’ (i.e., return to a home starting position), then follow the same pattern as before for the homing cycle. This process can take 30 seconds to 3 minutes depending on where the sweeping arms ended in the last program. Once all motors have homed out, the system is ready for the program to be entered on the monitor.

Section 2-2 Entering a Cage into the System

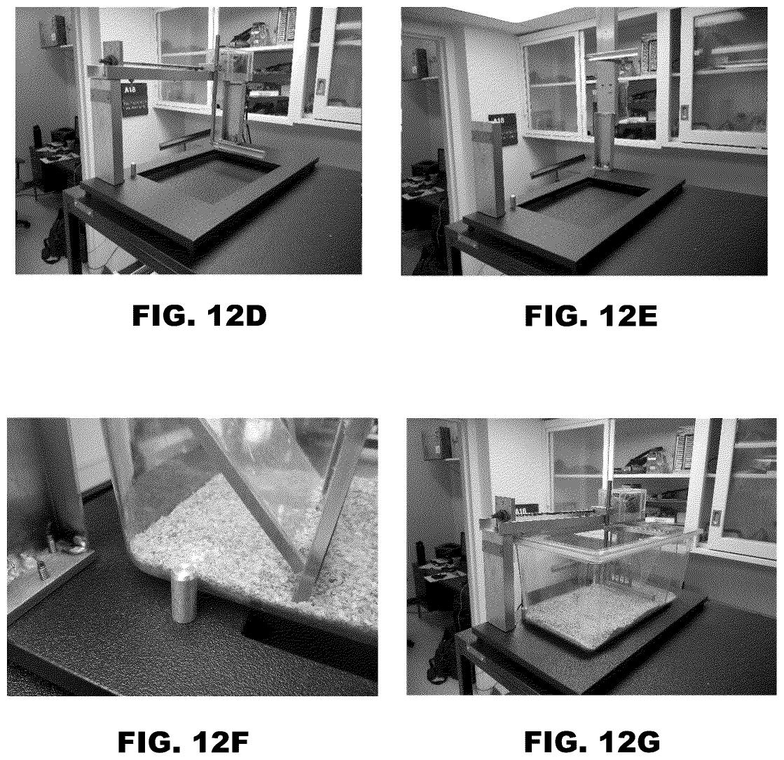

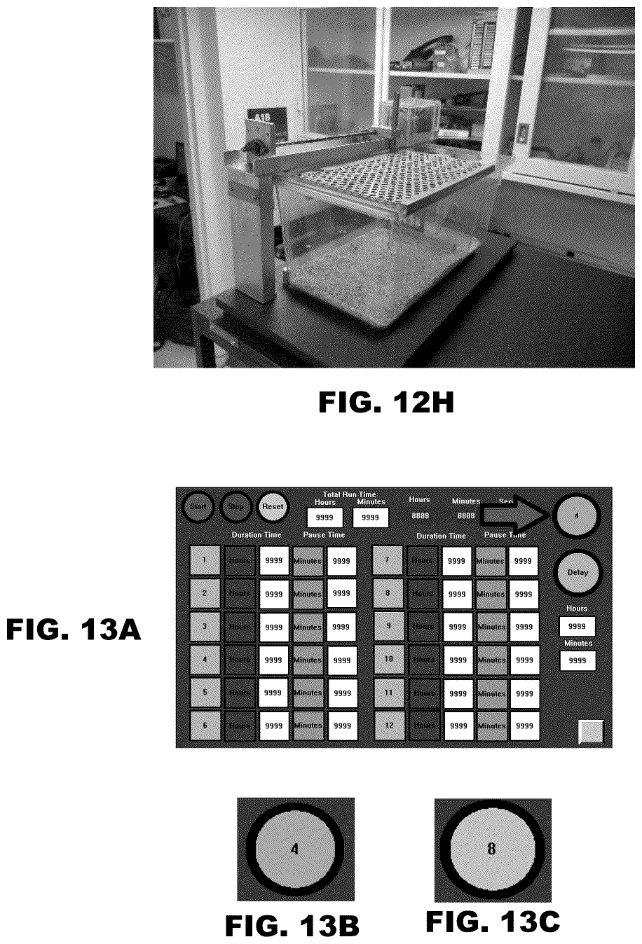

When the motors are in the home position, the user will remove the Metal Door from the Plastic Cage Cover, as shown progressively by FIGS. 12A, 12B, and 12C. Such removal allows the sweeping arm to enter the cage. Thereafter, the Support Arm is raised to allow the cage to slid into place, as progressively represented by FIGS. 12D and 12E. Next, the cage slides into the system until it hits the Cage Locating Pins, as represented by FIG. 12F. Thereafter, the Support Arm may be lowered to its lowered position, as represented by FIG. 12G. Such maneuver may require the user to move the cage side to side to help line up the Location Pins. Then, the Metal Door may be put back on the cage and the system is ready for experiments, as shown in FIG. 12H. Thus, such Figures illustrate that the subject Guide Rail is supported to be pivoted to a non-seated position for placement of the associated cage, and then pivoted into a seated position with the base portion of the sweep arm positioned to extend through the elongated slot and along the width of the cage floor.

### Section 2-3 Motor Selection Option

One of the options in programing is to determine how many motors are allowed to run during an experiment. This option can be found in the top righthand corner of the monitor, as shown in FIG. 13A. When the circle is orange with the number (e.g., four in the center (see FIG. 13B)), only motors 1 through 4 will run and motors 5 through 8 will stay stationary. But when pressed, the button will turn pink with the number eight in the center, as shown by FIG. 13C. With this option selected, all 8 motors will run simultaneously. This option can be turned off or on at any point in the program.

### Section 2-4 Delay Option

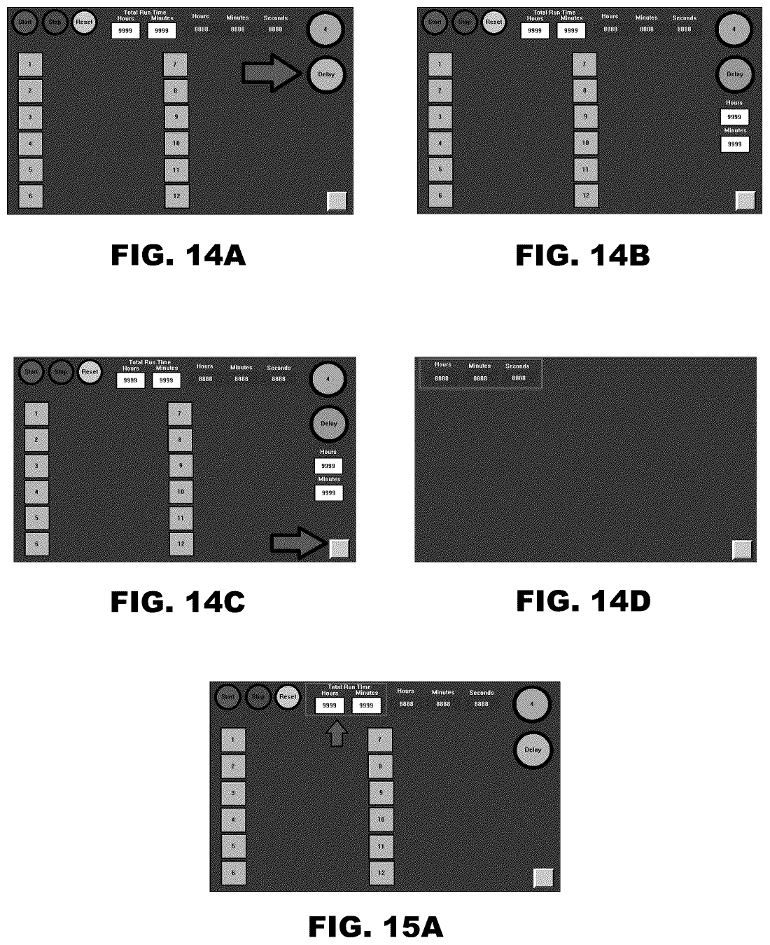

The Delay function, as referenced by FIG. 14A, allows a user to delay the start of a program for a defined amount of time. Pressing the Delay Button on the monitor screen allows activation of the Delay Function. The Button will turn green when on and reveal the area where the time for the Delay Function can be entered (as represented by FIG. 14B). The user then enters in the time of the desired delay period.

For a user to check whether the program has started, the Gold Page Button in the bottom righthand corner of the monitor screen (see FIG. 14C) is pushed to pull up the Delay timer page. The timer on this page states how long the delay has been running, as shown by FIG. 14D.

### Section 2-5 Entering a Program

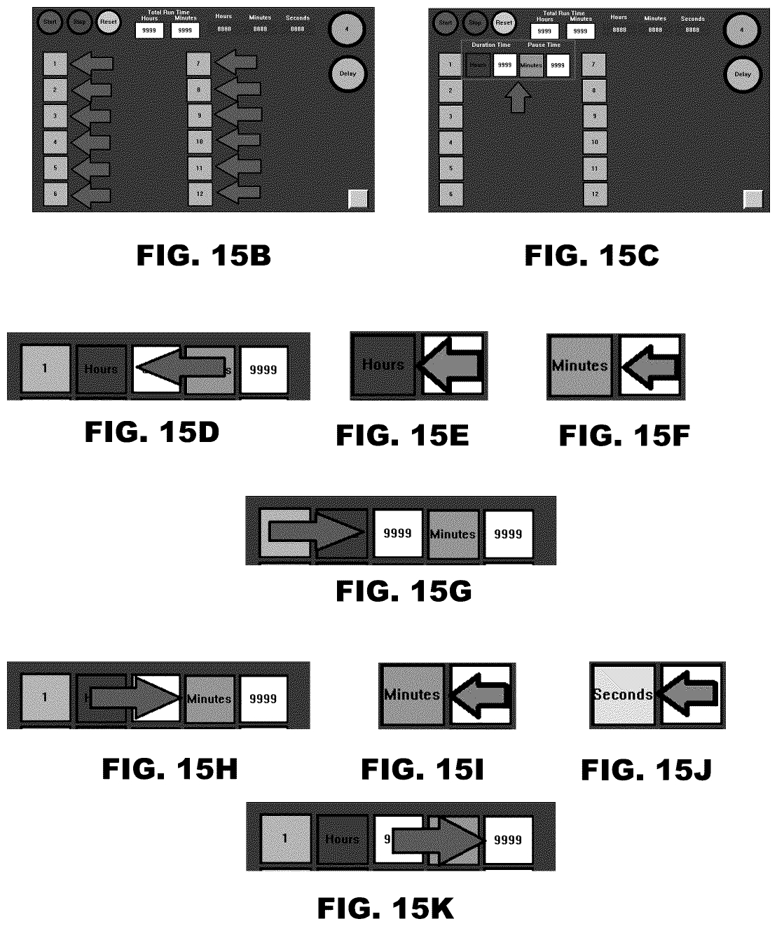

Step 1: A user may enter in the Total Run Time in the boxes in the top center of the Monitor screen. Time can be entered in as Hours and Minutes which will combine to create the total run time for the program, as represented in FIG. 15A.

Step 2: A user may select how many segments are needed by pressing the Duration Selection Buttons, as shown in FIG. 15B. When a Duration Selection Button is pressed, the options for that section pop up, as represented in FIG. 15C. The Total Run Time can be sectioned into 12 Durations. All the lengths of the Durations must equal the Total Run Time. If they do not equal each other, it could cause problems in the program.

Step 3: A user selects what Time Value is needed for the Duration Time. This can be either Hours or Minutes. The default value for each Duration is in Hours but can be changed to Minutes by pressing the Duration Time Value Selection Button shown in FIG. 15D. When pressed, the color of the button will change and will display what the value will be, as represented by FIGS. 15E and 15F.

Step 4: A user enters in the amount of time the Duration will last for into the Duration Length Entry Location, as represented by FIG. 15G. The numbers entered must be whole numbers or the program will not accept them. For example, if a user wants a Duration to last for an hour and a half, they put the Time Length in as 90 minutes, not 1.5 hours.

Step 5: The user selects what Time Value is needed for the Pause Time. The default values for a Pause is set in Minutes. The value can be changed by pressing on the Pause Time Value Selection Button shown in FIG. 15H. When pressed, the button will change colors and will display the new value, as represented by FIGS. 15I and 15J.

Step 6: A user may enter in the amount of time the Pause will last into the Pause Length Entry Location, as represented by FIG. 15K. The numbers entered must be whole numbers or the program will not accept them. For example, if a user wants a Pause to last for a minute and a half, the user must put the Pause Time in as 90 seconds, not 1.5 minutes.

Step 7: Steps 3 through 6 are repeated for each Duration needed for the program. When the program is fully entered, the user proceeds to the next section.

### Section 2-6 Starting and Stopping a Program

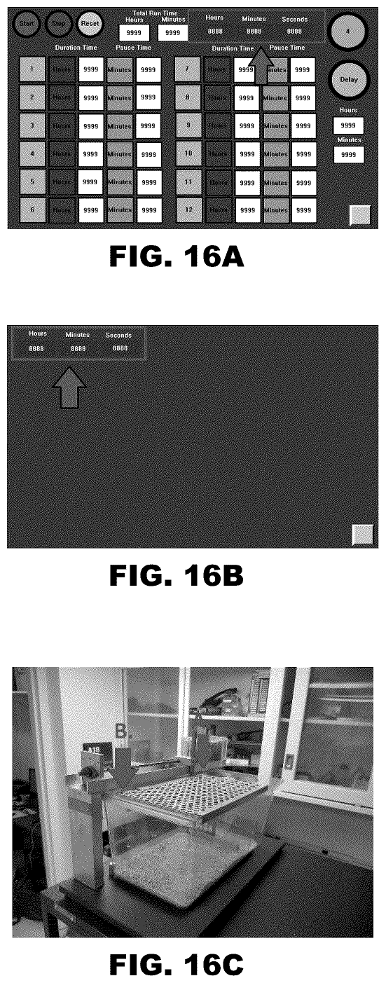

After checking all of the Values and the program is ready to Start, the user presses the Start Button. After pressing the Start Button, the user checks either the Program Running Timer or the Delay Timer if a delay is set. If either Timer is counting up, it signifies that the program has started, as referenced in FIGS. 16A and 16B.

If at any point the program must be stopped, the user presses the Stop Button. After pressing the Stop Button, the user checks the Program Running Timer. If it has stopped counting, the user has successfully stopped the program. If the Stop Button is pressed in the middle of a motor movement, the motor will continue to move until it reaches either Point A or Point B, as illustrated in FIG. 16C. The program may be restarted at any point by again pressing the Start Button.

### Section 2-7 Resetting a Program

If at any point a Program needs to be Reset to the beginning, the program must be Stopped first. Once the program is Stopped, the user presses the Reset Button to Reset the Program. To confirm that the Program has been Reset, the user checks the Program Running Timer. If it shows 0s across the board, the Program has been Reset.

### Section 2-8 End of Program/Shut Down Procedures

When a Program finishes, it will clear out the Program Running Timer but will keep all other values. If a Program needs to be run again, the user may do so by pressing the Start Button. The user should keep in mind that the Delay option turns itself off after it has finished its operation. Thus, in order to Delay the start of the Program, it must be turned back on, and the Delay Time reentered again. When the user is done with experiments and the system is no longer needed, the user should turn the power off. This can be done by either unplugging everything or by turning a power strip off if the Chamber is plugged into it. This is done to allow the system to rest and to remove all stress on the motors waiting for commands.

While certain embodiments of the disclosed subject matter have been described using specific terms, such description is for illustrative purposes only, and it is to be understood that changes and variations may be made without departing from the spirit or scope of the subject matter. For example, various and different times and time periods may be used in a given protocol, as compared to the exemplary times and time periods disclosed herewith.

What is claimed is:

1. Methodology for inducing sleep loss in animal test subjects, comprising:
providing an animal enclosure cage with a removable lid forming an elongated slot along the top of the enclosure cage;
mounting a controllably movable arm adjacent to the cage;
positioning the movable arm for projection into the cage through the elongated slot so that at least a portion of the arm is movable adjacent to the floor of the cage; and
controllably moving the movable arm along the cage floor so that an animal test subject in the cage is periodically moved to deprive sleep of the animal test subject.

2. Methodology according to claim 1, wherein the movable arm comprises an L-shaped arm having at least a portion thereof which is positioned to extend along the width of the cage floor.

3. Methodology according to claim 1, wherein the movable arm is mounted on a guide rail adjacent to the cage, and a controllable motor is attached to the arm for controllably driving the arm relative to the guide rail.

4. Methodology according to claim 3, further including programmably controlling the motor according to a sleep deprivation protocol to which an animal test subject in the cage is subjected.

5. Methodology according to claim 4, wherein the sleep deprivation protocol is no longer than six hours and the animal test subject has food and water available at all times in the cage.

6. Methodology according to claim 6, wherein the motor comprises a DC servo motor programmed according to the sleep deprivation protocol to sweep a portion of the arm along the cage floor over a controlled sweep duration and with a controlled pause between sweeps.

7. Methodology according to claim 7, wherein the controlled sweep duration and controlled pauses are variable in time.

8. Methodology according to claim 7, wherein the maximum speed at which the bar sweeps the entire cage is within 3 seconds.

9. Methodology according to claim 9, wherein the controlled pauses are set to vary in time between 0 seconds and 5 minutes.

10. Methodology according to claim 1, wherein:
the cage comprises a transparent standardized cage for observing test rats or test mice; and
the methodology further includes receiving and recording EEG/EMG telemetrically in test animals that have been implanted with sleep transmitters.

11. Methodology according to claim 1, further comprising a user controllably starting, stopping, and selectively interrupting the sleep deprivation protocol by controlling the motor operation.

12. Methodology according to claim 4, further comprising:
providing a plurality of respective animal enclosure cages each with removable lids with elongated slots along the tops of the respective enclosure cages, and each with controllably movable arms positioned for projection into its respective cage through its elongated; and
controllably moving in synchronization all of the movable arms along their respective cage floors so that an animal test subject in each respective cage is periodically moved to deprive sleep of the animal test subject, so that a corresponding plurality of animal test subjects in the respective plurality of cages may each be simultaneously subjected to the same sleep deprivation protocol.

13. A system for inducing sleep loss in animal test subjects, comprising:
a removable lid configured to fit on a preexisting animal enclosure cage, with an elongated slot formed along the top of the enclosure cage;
a guide rail situated adjacent to the cage;
a movable arm received on the guide rail for movement there along; and
a controllable motor for controllably driving the arm relative to the guide rail;
wherein the movable arm is an L-shaped arm having at least a portion thereof which is positioned to extend through the elongated slot and along the width of the cage floor while the arm is received on the guide rail, so that controllably moving the arm through operation of the controllable motor moves the movable arm along the cage floor so that an animal test subject in the cage is periodically moved to deprive sleep of the animal test subject.

14. A system according to claim 13, wherein:
the motor comprises a DC servo motor; and
the system further comprises a programmable logic control board for controlling the motor according to a sleep deprivation protocol to which an animal test subject in the cage is subjected.

15. A system according to claim 14, wherein the sleep deprivation protocol can be varied and the animal test subject has food and water available at all times in the cage.

16. A system according to claim 15, wherein the sleep deprivation protocol comprises the DC servo motor being programmed to sweep a portion of the arm along the cage floor over a controlled sweep duration and with a controlled pause between sweeps.

17. A system according to claim 16, wherein the controlled sweep duration and controlled pauses are variable in time.

18. A system according to claim 17, wherein:
the controlled pauses are set to vary in time between 0 seconds and 5 minutes; and
the maximum speed at which the bar sweeps the entire cage is within 3 seconds.

19. A system according to claim 13, wherein:
the preexisting animal enclosure cage comprises a transparent standardized cage for observing test rats or test mice; and
the system further includes a receiver for receiving and recording EEG/EMG telemetrically in test animals that have been implanted with sleep transmitters.

20. A system according to claim 14, further comprising:
a plurality of respective animal enclosure cages each with removable lids with elongated slots along the tops of the respective enclosure cages, and each with controllably movable arms positioned for projection into its respective cage through its elongated, and respective controllable motors for controllably driving its arm relative to its guide rail; and
wherein the programmable logic control board is connected for simultaneously controlling in synchronization each of the motors according to the sleep deprivation protocol so that an animal test subject in each respective cage is periodically moved to deprive sleep of the animal test subject, so that a corresponding plurality of animal test subjects in the respective plurality of cages may each be simultaneously subjected to the same sleep deprivation protocol.

21. An automated system for inducing sleep loss in rodent test subjects according to a programmed sleep deprivation protocol, comprising:
a rodent enclosure cage;
a removable lid configured to fit on the cage, with an elongated slot formed along the top of the cage;
a guide rail supported adjacent to the cage;
a movable sweep arm received on the guide rail for movement back and forth along the rail;
a controllable motor for controllably driving the sweep arm relative to the guide rail; and
a programmable motor controller for controlling operation of the motor according to a programmed sleep deprivation protocol;
wherein the sweep arm is L-shaped with a base portion thereof positioned to extend through the elongated slot and along the width of the cage floor while the arm is received on the guide rail, so that controllably moving the arm through operation of the controllable motor moves the movable arm along the cage floor so that a rodent test subject in the cage is periodically moved to deprive sleep of the rodent test subject according to the programmed sleep deprivation protocol.

22. An automated system according to claim 21, wherein:
the motor comprises a DC servo motor; and
the sleep deprivation protocol is varied during which the rodent test subject has food and water available at all times in the cage, and the protocol causes the arm base portion to sweep along the cage floor over a controlled sweep duration and with a controlled pause between sweeps.

23. An automated system according to claim 22, wherein the controlled sweep duration and controlled pauses are variable in time.

24. An automated system according to claim 23, wherein:
the controlled pauses are set to vary in time between 0 seconds and 5 minutes; and
the maximum speed at which the bar sweeps the entire cage is within 3 seconds.

25. An automated system according to claim 21, wherein:
the rodent enclosure cage comprises a transparent standardized cage for observing test rodents; and
the system further includes a receiver for receiving and recording EEG/EMG telemetrically in rodent test subjects that have been implanted with sleep transmitters.

26. An automated system according to claim 25, further comprising:
a plurality of respective rodent enclosure cages each with removable lids with elongated slots along the tops of the respective enclosure cages, and each with movable sweep arms received on guide rails and positioned to extend through its elongated slot and along the width of the cage floor while the arm is received on its guide rail, and respective controllable motors for controllably driving its arm relative to its guide rail; and
wherein the programmable motor controller is connected for simultaneously controlling in synchronization operation of each of the motors according to the programmed sleep deprivation protocol so that a rodent test subject in each respective cage is periodically moved to deprive sleep of the rodent test subject, so that a corresponding plurality of rodent test subjects in the respective plurality of cages may each be simultaneously subjected to the same programmed sleep deprivation protocol.

27. An automated system according to claim 21, wherein the guide rail is pivotally supported to be pivoted to a non-seated position for placement of the associated cage, and then pivoted into a seated position with the base portion of the sweep arm positioned to extend through the elongated slot and along the width of the cage floor.
